(19) United States
(12) Patent Application Publication
(10) Pub. No.: US 2023/0320698 A1
(43) Pub. Date: Oct. 12, 2023

(54) ULTRASONIC DIAGNOSTIC APPARATUS, METHOD FOR CONTROLLING ULTRASONIC DIAGNOSTIC APPARATUS, AND CONTROL PROGRAM FOR ULTRASONIC DIAGNOSTIC APPARATUS

(71) Applicant: KONICA MINOLTA, INC., Tokyo (JP)

(72) Inventor: Yoshihiro TAKEDA, Tokyo (JP)

(73) Assignee: KONICA MINOLTA, INC., Tokyo (JP)

(21) Appl. No.: 18/113,136

(22) Filed: Feb. 23, 2023

(30) Foreign Application Priority Data
Apr. 12, 2022 (JP) 2022-065764

(51) Int. Cl. A61B 8/00; A61B 8/08

(52) U.S. Cl. CPC A61B 8/461; A61B 8/085

(57) ABSTRACT

An ultrasonic diagnostic apparatus includes: a transmitter/receiver that causes an ultrasonic probe to transmit and receive an ultrasonic beam; a signal processor that generates an ultrasonic image on a basis of a reception signal acquired from the ultrasonic probe; a target identifier that performs segmentation processing based on a structure type on the ultrasonic image to generate a likelihood image indicating an existence region of a target in the ultrasonic image; and a likelihood image synthesizer that synthesizes the likelihood images of a plurality of the ultrasonic images generated by ultrasonic scanning using the ultrasonic beams having steer angles different from each other to generate a space compound likelihood image.

The entire disclosure of Japanese patent Application No. 2022-065764, filed on Apr. 12, 2022, is incorporated herein by reference in its entirety.

### BACKGROUND

### Technological Field

The present invention relates to an ultrasonic diagnostic apparatus, a method for controlling an ultrasonic diagnostic apparatus, and a control program for an ultrasonic diagnostic apparatus.

### Description of the Related Art

Conventionally, as one of medical image diagnostic apparatuses, there has been known an ultrasonic diagnostic apparatus that transmits an ultrasonic wave toward a subject, receives a reflected wave of the ultrasonic wave, and performs predetermined signal processing on a reception signal to visualize a shape, a property, or dynamics inside the subject as an ultrasonic image. Since the ultrasonic diagnostic apparatus can acquire an ultrasonic image by a simple operation of applying an ultrasonic probe to a body surface or inserting the ultrasonic probe into a body, the ultrasonic diagnostic apparatus is safe, and a burden on a subject is small.

The ultrasonic diagnostic apparatus is used, for example, when a target region is treated by inserting a puncture needle into a body of a subject under an ultrasonic guide. In such treatment, a practitioner such as a doctor can insert the puncture needle and perform the treatment while confirming a treatment target region by viewing an ultrasonic image obtained by the ultrasonic diagnostic apparatus.

When the treatment is performed wider the ultrasonic guide, in order to accurately grasp a position of the treatment target region, it is preferable that the target region is clearly reflected in the ultrasonic image (B-mode image). For example, in a nerve block in which local anesthesia is performed by puncturing a peripheral nerve directly or around the peripheral nerve, a nerve into which an anesthetic is injected, a blood vessel in which the anesthetic should not be erroneously injected, or the like can be a target. Further, in the nerve block, the practitioner visually distinguishes between the nerve and the blood vessel on the ultrasonic image and pays attention not to puncture the blood vessel, but high skill and abundant experience are required.

From such a background, in recent years, a technique of identifying a target in an ultrasonic image and providing a display image of the ultrasonic image to a practitioner (hereinafter also referred to as “user”) in a mode capable of identifying a region of the target has also been proposed (see, for example, JP 2019-508072 W and JP 2021-058232 A).

FIG. 1 is a diagram illustrating an example of an image processing method of an ultrasonic image according to a conventional technique.

In the image processing method according to the conventional technique, for example, a target (for example, nerve tissue) in an ultrasonic image is identified using an identification model trained by machine learning, and a likelihood image in which a region having a high likelihood (that is, a certainty factor) as an existence region of the target in the ultrasonic image is distinguished from a region having a low likelihood (that is, a certainty factor) is generated (also referred to as segmentation processing). Then, in the image processing method according to the conventional technique, color information (hue, saturation, and lightness) is added to each pixel of the ultrasonic image on the basis of a pixel value of the ultrasonic image and a pixel value of the likelihood image using the color map, and at least one of the hue, saturation, and lightness of the ultrasonic image is changed to generate a display image to be provided for a user.

Note that “likelihood” of a target is an index indicating likelihood of being a target, and a likelihood of the existence region of the target is large and a likelihood of a region of a non-target is small. In addition. “likelihood image” is an image representing a distribution of the likelihood of the target (that is, the existence region of the target) corresponding to the entire ultrasonic image.

Meanwhile, in this type of ultrasonic diagnostic apparatus, the inventors of the present application have studied application of a space compound method for the purpose of providing a user with a high-quality ultrasonic image and more accurately presenting a region of a target in the ultrasonic image (including a target such as a puncture needle that urges a user to pay attention to an existence region in addition to a puncture target such as nerve tissue; the same applies hereafter). The space compound method is a method of generating a plurality of frame images by transmitting ultrasonic beams from directions different from each other toward the same part in a subject, and synthesizing the plurality of frame images to generate one space compound image.

FIG. 2 is a diagram for explaining a general space compound method.

In the space compound method, for example, as illustrated in FIG. 2, an ultrasonic image B generated by ultrasonic scanning using an ultrasonic beam having a steer angle of 0 degrees, an ultrasonic image A generated by ultrasonic scanning using an ultrasonic beam having a steer angle of −θ degrees, and an ultrasonic image C generated by ultrasonic scanning using an ultrasonic beam having a steer angle of +θ degrees are repeatedly generated in the same order in a three-frame cycle. Every time one frame of reception data is acquired, ultrasonic images for three frames obtained by combining ultrasonic images for the two preceding frames are synthesized to generate a space compound image Sy. As a result, the space compound image Sy obtained by synthesizing the ultrasonic images A, B, and C corresponding to the three types of steering angles is always updated.

According to such a space compound method, by synthesizing a plurality of frame images generated by transmitting ultrasonic beams from directions different from each other, speckle noise generated due to scattered waves from an infinite number of scattering sources present in a subject can be reduced, and acoustic noise such as shading can be reduced.

However, the space compound method according to the conventional technique has several problems that make an existence region of a target uncertain.

FIG. 3 is a diagram for explaining a motion artifact which is one of the problems of the space compound method according to the conventional technique.

In general, in ultrasonic inspection, a user moves an ultrasonic probe along a body surface of a subject in order to search for a treatment target (for example, nerve tissue) or the like present in the subject. Therefore, an imaging position of a frame image in each direction of a target to be synthesized in space compound processing is shifted. As a result, a space compound image generated by synthesizing the frame image in each direction becomes unclear, and it is difficult to identify a target (nerve tissue HT in FIG. 3) from the space compound image. Note that such a motion artifact occurs not only due to movement of the ultrasonic probe but also due to movement of the tissue (for example, the heart) itself in the subject.

FIG. 4 is a diagram for describing an increase in identification difficulty level of a structure having acoustic reflection anisotropy (hereinafter simply referred to as “anisotropy”) with respect to an ultrasonic beam, which is another problem of the space compound method according to the conventional technique.

In general, a structure having no anisotropy with respect to an ultrasonic beam, such as nerve tissue (HT in FIG. 4), and a structure having anisotropy with respect to the ultrasonic beam, such as a puncture needle (QT in FIG. 4), are mixed as a target for attracting user's attention to an existence position in ultrasonic inspection. An ultrasonic wave is generally reflected at a boundary having a difference in acoustic impedance. The wave is more strongly reflected as it hits a boundary surface at an angle close to 90 degrees, and a clear reflected ultrasonic wave is obtained. Therefore, a structure that induces reflected ultrasonic waves in various directions with respect to an incident ultrasonic beam, such as nerve tissue, does not depend on a beam direction of the ultrasonic beam, and thus there is no possibility that a drawing state becomes unclear on a space compound image. However, in a case of the puncture needle, when the beam direction of the ultrasonic beam is a direction orthogonal to an extending direction of the puncture needle, the puncture needle is clearly visualized in an ultrasonic image, but when the beam direction of the ultrasonic beam is a direction parallel to the extending direction of the puncture needle, the puncture needle is hardly visualized in the ultrasonic image.

That is, in a method of generating a space compound image by simply averaging a frame image in each direction as in the space compound method according to the conventional technique, an image of a structure having anisotropy such as a puncture needle becomes unclear as a result of image synthesis, and it becomes more difficult to identify such a structure in the space compound image than usual.

FIG. 5 is a diagram for describing an increase in identification difficulty level of a structure existing at an end of an image, which is another problem of the space compound method according to the conventional technique.

In general, in space compound processing, an ultrasonic image generated by ultrasonic scanning using an ultrasonic beam in an outer transmission direction (the ultrasonic image A and the ultrasonic image C in FIG. 2) is trimmed in accordance with an image area of an ultrasonic image generated by ultrasonic scanning using an ultrasonic beam having a steer angle of 0 degrees, and then image synthesis of these images is performed.

Usually, the more a target (the nerve tissue HT in FIG. 3) appears in the ultrasonic image over entire circumference, the easier target identification processing becomes. Ina case where the target exists at an edge of the image and is depicted in a partially missing state, an identification difficulty level of the target increases. That is, identification is possible in an ultrasonic image generated by ultrasonic scanning using an ultrasonic beam in an outer transmission direction (see an ultrasonic image A in FIG. 5), but is more difficult than usual in a space compound image generated by simply averaging a frame image in each direction.

### SUMMARY

The present disclosure has been made in view of the above problems, and an object of the present disclosure is to provide an ultrasonic diagnostic apparatus, a method for controlling an ultrasonic diagnostic apparatus, and a control program for an ultrasonic diagnostic apparatus that enable improvement in accuracy of a likelihood image indicating a target region in ultrasonic image diagnosis using a space compound method.

To achieve the above mentioned object, according to an aspect of the present invention, an ultrasonic diagnostic apparatus reflecting one aspect of the present invention comprises: a transmitter/receiver that causes an ultrasonic probe to transmit and receive an ultrasonic beam; a signal processor that generates an ultrasonic image on a basis of a reception signal acquired from the ultrasonic probe; a target identifier that performs segmentation processing based on a structure type on the ultrasonic image to generate a likelihood image indicating an existence region of a target in the ultrasonic image; and a likelihood image synthesizer that synthesizes the likelihood images of a plurality of the ultrasonic images generated by ultrasonic scanning using the ultrasonic beams having steer angles different from each other to generate a space compound likelihood image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of an image processing method of an ultrasonic image according to a conventional technique;

FIG. 2 is a diagram for explaining a general space compound method;

FIG. 3 is a diagram for explaining a motion artifact which is one problem of the space compound method according to the conventional technique;

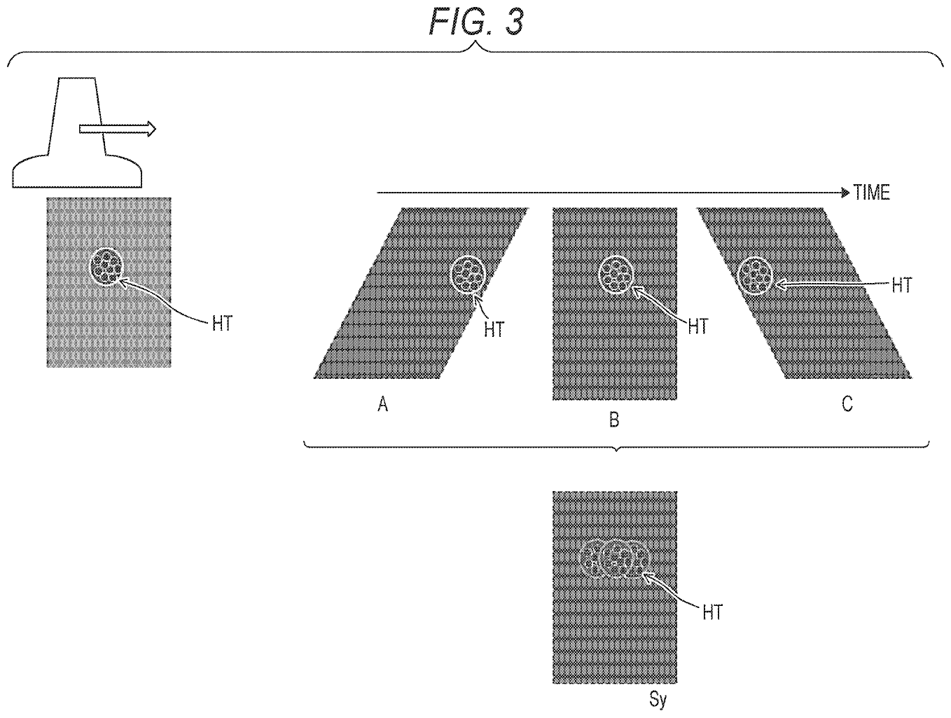

FIG. 4 is a diagram for describing an increase in identification difficulty level of a structure having acoustic reflection anisotropy with respect to an ultrasonic beam, which is another problem of the space compound method according to the conventional technique:

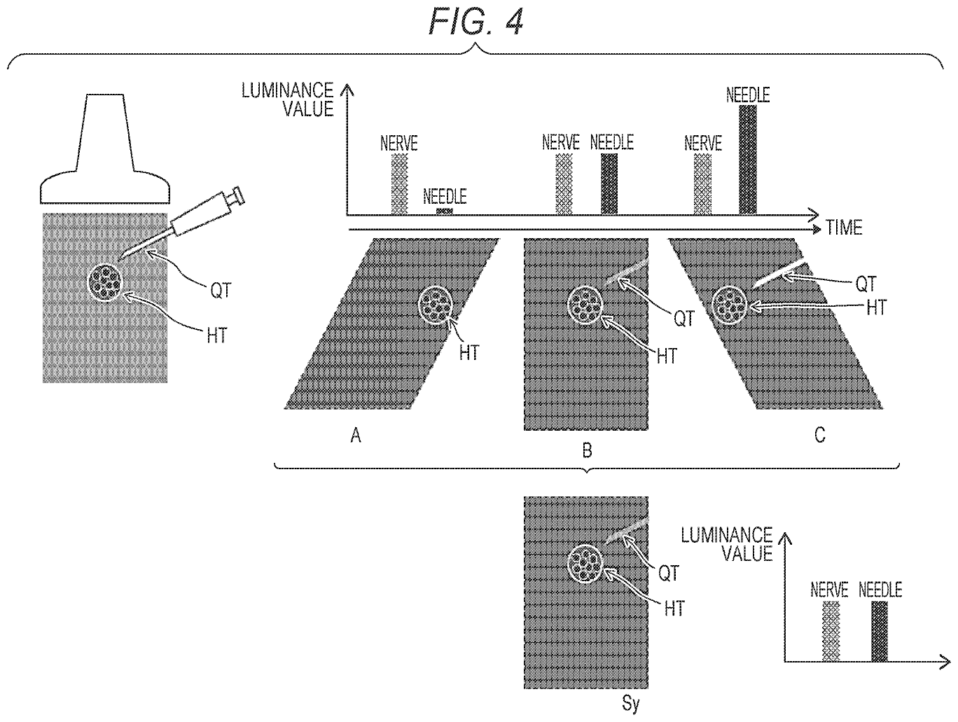

FIG. 5 is a diagram for describing an increase in identification difficulty level of a structure existing at an end of an image, which is another problem of the space compound method according to the conventional technique;

FIG. 6 is a diagram illustrating an example of an appearance of an ultrasonic diagnostic apparatus according to an embodiment of the present invention;

FIG. 7 is a block diagram illustrating a main part of a control system of the ultrasonic diagnostic apparatus:

FIG. 8 is a diagram illustrating a detailed configuration of an image processing unit;

FIG. 9 is a diagram for explaining processing performed by a target identifier,

FIG. 10 is a diagram for describing processing performed by a likelihood image synthesizer, and

FIG. 11 is a diagram illustrating an example of an image synthesis method according to an identification target stored in an image synthesis method data table.

### DETAILED DESCRIPTION OF EMBODIMENTS

Hereinafter, one or more embodiments of the present invention will be described with reference to the drawings. However, the scope of the invention is not limited to the disclosed embodiments. Note that, in the present specification and the drawings, components having substantially the same function are denoted by the same reference numerals, and redundant description is omitted.

[Overall Configuration of Ultrasonic Diagnostic Apparatus]

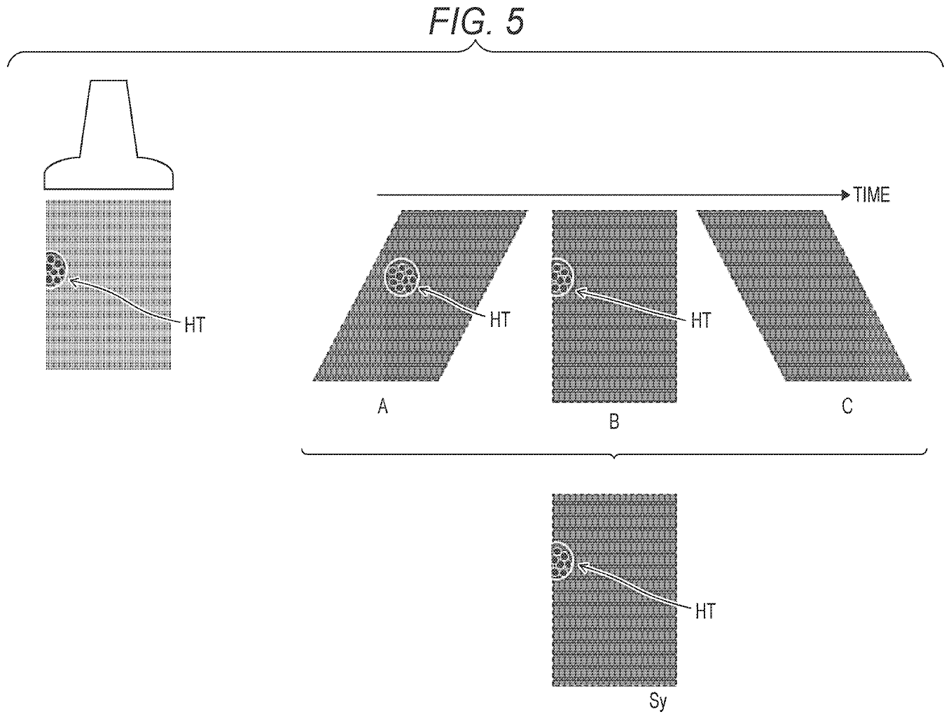

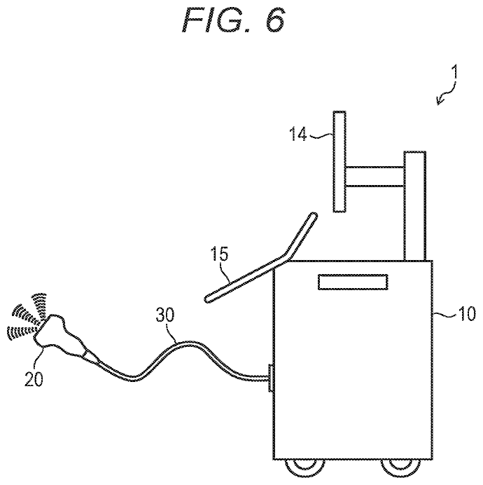

Hereinafter, an overall configuration of an ultrasonic diagnostic apparatus (hereinafter “ultrasonic diagnostic apparatus 1”) according to an embodiment of the present invention will be described with reference to FIGS. 5 and 6. Note that the ultrasonic diagnostic apparatus t according to the present embodiment is used, for example, to visualize a shape, a property, or dynamics in a subject as an ultrasonic image and perform image diagnosis.

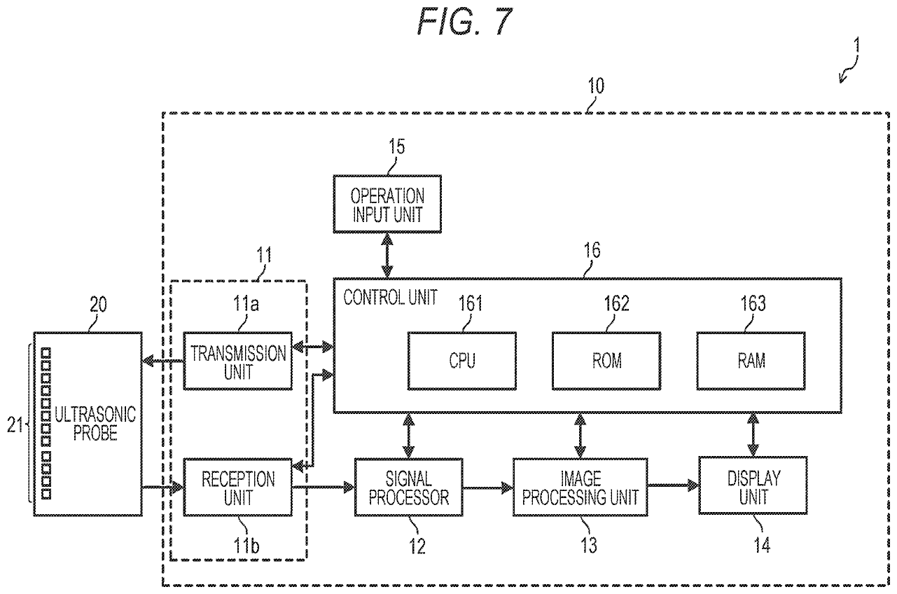

FIG. 6 is a diagram illustrating an example of an appearance of the ultrasonic diagnostic apparatus 1 according to the present embodiment. FIG. 7 is a block diagram illustrating a main part of a control system of the ultrasonic diagnostic apparatus 1 according to the present embodiment.

The ultrasonic diagnostic apparatus 1 is used to visualize a shape, a property, or dynamics in a subject as an ultrasonic image and perform image diagnosis. For example, the ultrasonic diagnostic apparatus 1 has a function of visually presenting an existence region of a target as puncture support information so as to be superimposed on a B-mode image when the subject is punctured and an anesthetic is injected into a nerve or around the nerve to perform a nerve block.

Note that, in the present embodiment, for example, nerve tissue for grasping a region into which the puncture needle is to be inserted and a puncture needle itself can be “target” that urges a user to pay attention to the existence region. However, setting of the target can be arbitrarily changed according to a use mode of the ultrasonic diagnostic apparatus of the user. A nerve may be treated as the target, and structures other than the nerve such as a blood vessel, a bone, and a muscle fiber ray be treated as a non-target. Alternatively, the nerve and the blood vessel that should not be inserted with the puncture needle may be treated as the target, and structures other than the nerve and the blood vessel may be treated as the non-target.

The ultrasonic diagnostic apparatus 1 includes an ultrasonic diagnostic apparatus main body 10 and an ultrasonic probe 20. The ultrasonic diagnostic apparatus main body 10 and the ultrasonic probe 20 are connected via, for example, a cable 30.

The ultrasonic probe 20 transmits an ultrasonic wave to the subject, receives an ultrasonic echo reflected in the subject, converts the ultrasonic echo into a reception signal, and transmits the reception signal to the ultrasonic diagnostic apparatus main body 10. Any probe of a convex type, a linear type, a sector type, or the like can be applied to the ultrasonic probe 20.

The ultrasonic probe 20 includes an array transducer 21 including a plurality of piezoelectric transducers arranged in an array shape, and a channel switching unit (not illustrated) for individually switching on/off of a driving state of each of the plurality of piezoelectric transducers constituting the array transducer 21.

The array transducer 21 includes the plurality of piezoelectric transducers arranged in the array shape along a scanning direction, for example. The on/off of the driving states of the plurality of piezoelectric transducers constituting the array transducer 21 is sequentially controlled to switch along the scanning direction individually or in units of blocks under the control of the channel switching unit by a control unit 16. That is, the plurality of piezoelectric transducers individually or in units of blocks converts a voltage pulse generated by a transmitter/receiver 11 into an ultrasonic beam and transmits the ultrasonic beam into the subject. Also, the plurality of piezoelectric transducers receives a reflected wave beam generated by reflection of the ultrasonic beam in the subject, converts the reflected wave beam into an electric signal, and outputs the electric signal to the transmitter/receiver 11. As a result, the ultrasonic probe 20 transmits and receives ultrasonic waves so as to scan the inside of the subject.

The ultrasonic diagnostic apparatus main body 10 includes the transmitter/receiver 11, a signal processor 12, an image processing unit 13, a display unit 14, an operation input unit 15, and the control unit 16.

The transmitter/receiver 11 is a transmission/reception circuit that causes the ultrasonic probe 20 to transmit and receive an ultrasonic wave.

The transmitter/receiver 11 includes a transmission unit 11a that generates a voltage pulse (hereinafter referred to as “drive signal”) and transmits the voltage pulse to each piezoelectric transducer of the ultrasonic probe 20, and a reception unit 11b that performs reception processing of an electric signal (hereinafter referred to as “reception signal”) related to a reception beam generated by each piezoelectric transducer of the ultrasonic probe 20. Then, the transmission unit 11a and the reception unit 11b each execute an operation of causing the ultrasonic probe 20 to transmit and receive an ultrasonic wave under the control of the control unit 16.

The transmission unit 11a includes, for example, a pulse oscillator, a pulse setting unit, and the like provided for each channel connected to the ultrasonic probe 20. The transmission unit 11a adjusts a voltage pulse generated by the pulse oscillator to a voltage pulse having voltage amplitude, pulse width, and timing set in the pulse setting unit, and transmits the voltage pulse to the array transducer 21. Note that the transmission unit 11a appropriately sets a delay time for each channel such that the ultrasonic wave output from each piezoelectric transducer of the ultrasonic probe 20 is focused in a beam shape in a predetermined direction, and supplies a drive signal to each piezoelectric transducer.

The reception unit 11b includes, for example, a preamplifier, an AD converter, and a reception beamformer. The preamplifier and the AD convener are provided for each channel connected to the ultrasonic probe 20, and amplify a weak reception signal and convert the amplified reception signal (analog signal) into a digital signal. The reception beamformer combines the plurality of reception signals into one by phasing addition of the reception signal (digital signal) of each channel, and outputs the combined reception signal to the signal processor 12. In the reception beamformer, for example, a delay time is appropriately set for each channel so as to focus an ultrasonic echo from a predetermined direction, and a plurality of reception signals is combined into one and output to the signal processor 12. In addition, in the reception beamformer, dynamic reception focus control is performed so that a reception focus point is continuously moved in a deep direction from the vicinity of an ultrasonic emission surface of the ultrasonic probe 20.

The signal processor 12 performs detection (envelope detection) of sound ray data input from the reception unit 11b to acquire a signal, and performs logarithmic amplification, filtering (for example, low-frequency transmission, smoothing, and the like), enhancement processing, and the like as necessary. Then, the signal processor 12 sequentially accumulates the reception signal at each scanning position in a frame memory, and generates two-dimensional data including sampling data (for example, signal strength of the reception signal) at each position in a cross section along the scanning direction and a depth direction. For example, the signal processor 12 converts the signal strength of the reception signal at each position of the two-dimensional data into a pixel value, and generates data of an ultrasonic image (hereinafter abbreviated as “ultrasonic image”) for B-mode display of one frame. Them the signal processor 12 generates such an ultrasonic image every time the transmitter/receiver 11 scans the inside of the subject.

Note that the signal processor 12 may include an orthogonal detection processing unit, an autocorrelation calculation unit, or the like so that an ultrasonic image related to a Doppler image can be generated.

The image processing unit 13 applies space compound processing to the ultrasonic image generated by the signal processor 12 and synthesizes a plurality of ultrasonic images generated by ultrasonic scanning using ultrasonic beams having steer angles different from each other to generate one ultrasonic image (hereinafter referred to as “space compound ultrasonic image”) as an image for display.

In addition, the image processing unit 13 performs segmentation processing based on a structure type on the ultrasonic image generated by the signal processor 12 to generate a likelihood image indicating an existence region of a target. Then, the image processing unit 13 synthesizes the likelihood images of the plurality of ultrasonic images generated by the ultrasonic scanning using the ultrasonic beams having steer angles different from each other to generate one likelihood image (hereinafter referred to as “space compound likelihood image”) as an image for display.

Note that the transmitter/receiver 11, the signal processor 12, and the image processing unit 13 include, for example, dedicated or general-purpose hardware (that is, an electronic circuit) corresponding to each processing such as an application specific integrated circuit (ASIC) or a field-programmable gate array (FPGA), and implement each function in cooperation with the control unit 16. However, some or all of these may be realized by a digital signal processor (DSP), a central processing unit (CPU), a general-purpose graphics processing units (GPGPU), or the like performing arithmetic processing according to a program.

The display unit 14 is, for example, a display such as a liquid crystal display (LCD). The display unit 14 acquires display image data from the image processing unit 13 and displays the display image data.

The operation input unit 15 is, for example, a keyboard, a mouse, or the like, and acquires an operation signal input by a user. For example, the operation input unit 15 can set a type of the ultrasonic probe 20, a type of the subject (that is, a type of biological tissue), depth of an imaging target in the subject, an imaging mode (for example, the B mode, a C mode, or an E mode), or the like on the basis of the operation input by the user.

The control unit 16 performs overall control of the ultrasonic diagnostic apparatus 1 by controlling the transmitter/receiver 11, the signal processor 12, the image processing unit 13, the display unit 14, and the operation input unit 15 according to their functions.

The control unit 16 includes, for example, a central processing unit (CPU) as an arithmetic/control device, a read only memory (ROM) and a random access memory (RAM) as main storage devices, and the like. The ROM stores a basic program and basic setting data. The CPU reads a program corresponding to a processing content from the ROM, develops the program in the RAM, and executes the developed program, thereby centrally controlling an operation of each functional block of the ultrasonic diagnostic apparatus main body 10.

Note that, in the present embodiment, the functions of the functional blocks are implemented by cooperation of the hardware constituting the functional blocks and the control unit 16. However, some or all of the functions of the functional blocks may be implemented by the control unit 16 executing a program.

Note that the control unit 16 determines ultrasonic transmission/reception conditions (for example, an opening condition, a focusing point, a transmission waveform, a center frequency or a band, and apodize) in the ultrasonic probe 20 on the basis of the type (for example, the convex type, the sector type, the linear type, or the like) of the ultrasonic probe 20, the depth of the imaging target in the subject, the imaging mode (for example, the B mode, the C mode, or the E mode), and the like set by the operation input unit 15. Then, the control unit 16 operates the transmitter/receiver 11 according to the ultrasonic transmission/reception conditions in the ultrasonic probe 20.

[Detailed Configuration of Image Processing Unit 13]

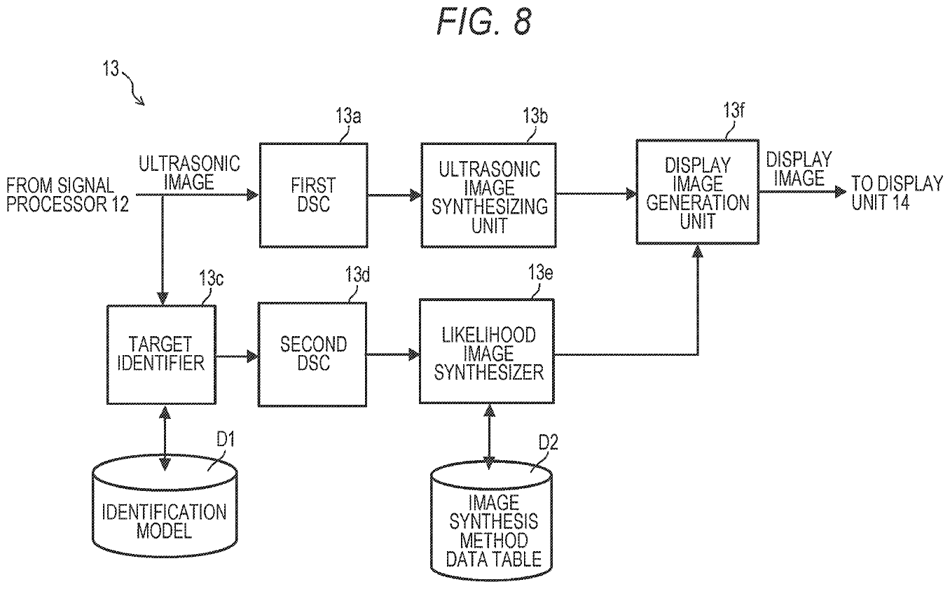

FIG. 8 is a diagram illustrating a detailed configuration of the image processing unit 13 according to the present embodiment.

The image processing unit 13 according to the present embodiment includes a first digital scan converter (DSC) 13a, an ultrasonic image synthesizing unit 13b, a target identifier 13c, a second digital scan convener (DSC) 13d, a likelihood image synthesizer 13e, and a display image generation unit 13f.

The first DSC 13a performs coordinate conversion processing and pixel interpolation processing according to the type of the ultrasonic probe 20 on the ultrasonic image generated by the signal processor 12, and converts data of the ultrasonic image into data of a display image according to a television signal scanning method of the display unit 14.

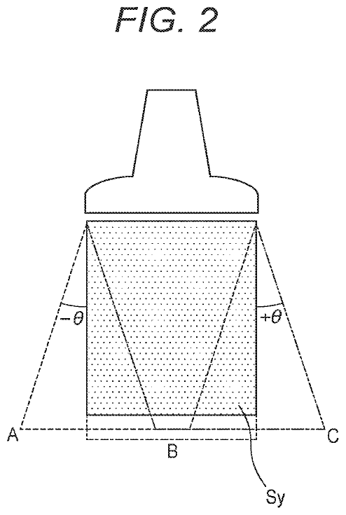

As described above with reference to FIG. 2, the ultrasonic image synthesizing unit 13b synthesizes a plurality of ultrasonic images generated by ultrasonic scanning using ultrasonic beams having steer angles different from each other to generate one space compound ultrasonic image.

In the present embodiment, as an example, the steer angle of the ultrasonic beam transmitted from the ultrasonic probe 20 is controlled under the control of the control unit 16. As illustrated in FIG. 2, an ultrasonic image B generated by ultrasonic scanning using an ultrasonic beam with a steer angle of 0 degrees, an ultrasonic image A generated by ultrasonic scanning using an ultrasonic beam with a steer angle of −θ degrees, and an ultrasonic image C generated by ultrasonic scanning using an ultrasonic beam with a steer angle of +θ degrees are repeatedly generated in the same order in a three-frame cycle. Each time the ultrasonic image for one frame is acquired, the ultrasonic image synthesizing unit 13b synthesizes ultrasonic images for three frames obtained by combining the ultrasonic images for the two preceding frames to generate a space compound ultrasonic image Sy.

At this time, for example, the ultrasonic image synthesizing unit 13b trims an outer region that does not overlap with the ultrasonic image B generated by the ultrasonic scanning using the ultrasonic beam with the steer angle of 0 degrees of the ultrasonic image A generated by the ultrasonic scanning using the ultrasonic beam with the steer angle of −θ degrees and the ultrasonic image C generated by the ultrasonic scaring using the ultrasonic beam with the steer angle of +θ degrees. The ultrasonic image synthesizing unit unifies coordinate systems of the ultrasonic image A, the ultrasonic image B, and the ultrasonic image C, and thereafter, synthesizes the ultrasonic image A, the ultrasonic image B, and the ultrasonic image C by a method of adding/averaging portions where B-mode image signals acquired from the same position overlap with each other.

Note that the number of frames of the ultrasonic image to be synthesized by the ultrasonic image synthesizing unit 13b may be other than three.

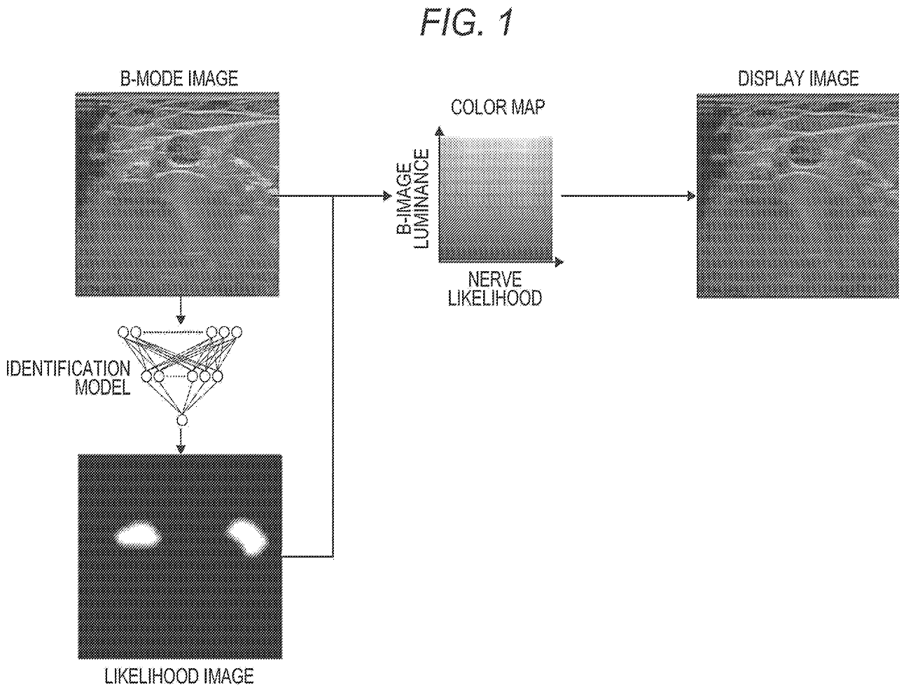

As described above with reference to FIG. 1, the target identifier 13c performs segmentation processing based on a structure type on the ultrasonic image generated by the signal processor 12 using an identification model D1 to generate a likelihood image indicating an existence region of a target in the ultrasonic image (image indicating a likelihood distribution of a target in the ultrasonic image). That is, the target identifier 13c identifies a target (for example, nerve tissue or a puncture needle) in the ultrasonic image.

Here, the identification model D1 is, for example, a neural network (for example, a convolutional neural network), and is subjected to learning processing in advance using a known machine learning algorithm (for example, an error backpropagation method) so as to extract a feature amount of an ultrasonic image from an input ultrasonic image and output a likelihood distribution of a target in the ultrasonic image. The identification model is stored in advance in a storage unit included in the image processing unit 13. The identification model D1 is typically constructed by supervised learning using teacher data configured by a data set in which an ultrasonic image and a likelihood distribution of a target are associated with each other. Note that, for an example of learning processing of the identification model D1, refer to, for example, JP 2021-058232 A, which is a prior application of the applicant of the present application.

The identification model D1 is subjected to learning processing so as to identify at least one structure type of nerve tissue, blood vessel tissue, muscle tissue, fascia tissue, tendon tissue, or a puncture needle from the ultrasonic image, for example. The identification model D1 may be separately prepared for each structure type, or one identification model D1 may be configured to identify a plurality of structure types. Similarly, the target identifier 13c may switch a type of the identification model D1 according to a type of the target to be identified.

That is, the identification model D1 calculates a likelihood of a target for each pixel or each pixel block (meaning a pixel group including a plurality of pixels) in association with each pixel region in the ultrasonic image, and outputs a likelihood distribution (that is, a likelihood image) of the target corresponding to the entire input ultrasonic image. The identification model D1 according to the present embodiment is configured to output a likelihood of a target corresponding to a central pixel block of an input ultrasonic image having a predetermined size. Then, the target identifier 13c switches the input image for the identification model D1 so as to scan the entire ultrasonic image for each predetermined size by raster scan, thereby outputting the likelihood distribution (that is, the likelihood image) of the target in the entire ultrasonic image. At this time, the target identifier 13c outputs the likelihood distribution of the target from the input image by, for example, forward propagation processing of the identification model D1 (neural network).

The likelihood image generated by the target identifier 13c is, for example, data in which a likelihood of any value in a range of 0 to 1 is calculated for each pixel region corresponding to each pixel region of the ultrasonic image (see FIG. 1). For example, such a likelihood image may indicate a likelihood distribution of one type of target (for example, nerve tissue) in the entire ultrasonic image, or may indicate a likelihood distribution of each of a plurality of types of targets (for example, nerve tissue and a puncture needle) in the entire ultrasonic image. In addition, size (that is, the number of pixels) of the likelihood image may be the same size as size of the ultrasonic image, or may be scaled down from the size of the ultrasonic image.

Note that the identification model D1 used by the target identifier 13c may be an identification model other than the neural network, and a support vector machine (SVM), a k-nearest neighbor algorithm, a random forest, a combination thereof, or the like may be used. This type of identification model is useful in that a highly robust identification device can be configured since the identification model is autonomously optimized so that a feature of a pattern to be identified can be extracted by performing learning processing and the pattern to be identified can be accurately identified from data on which noise or the like is superimposed.

The second DSC 13d performs coordinate conversion processing and pixel interpolation processing according to the type of the ultrasonic probe 20 on the likelihood image generated by the target identifier 13c, and converts data of the likelihood image into data of a display image according to the television signal scanning method of the display unit 14.

The likelihood image synthesizer 13e synthesizes likelihood images obtained from a plurality of ultrasonic images generated by ultrasonic scanning using ultrasonic beams having steer angles different from each other to generate a space compound likelihood image.

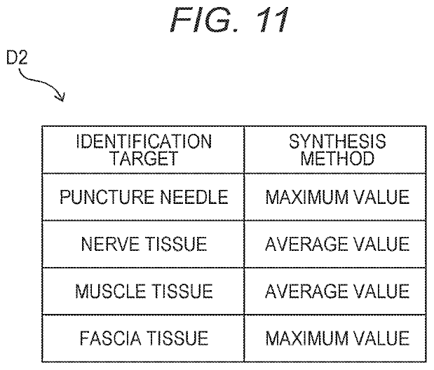

Basically, similarly to the synthesis processing of the ultrasonic image synthesizing unit 13b, the likelihood image synthesizer 13e unifies coordinate systems of the plurality of likelihood images, and then synthesizes the plurality of likelihood images by averaging likelihoods of the plurality of likelihood images at the same position or the like. However, the likelihood image synthesizer 13e refers to an image synthesis method data table D2 stored in advance in the storage unit (not illustrated) included in the image processing unit 13, and synthesizes the plurality of likelihood images using an image synthesis method set for each structure type (see FIG. 11).

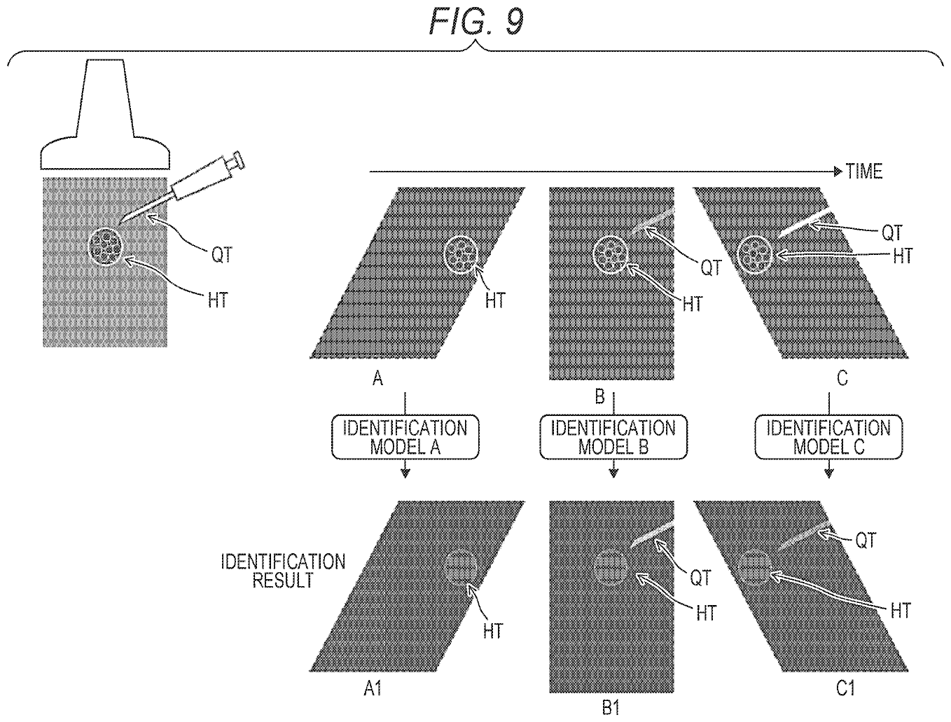

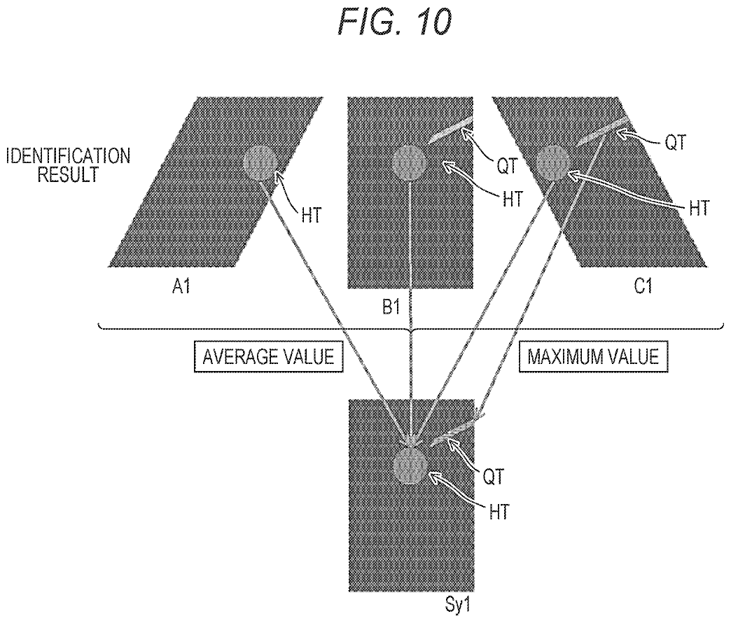

FIG. 9 is a diagram for explaining processing performed by the target identifier 13c according to the present embodiment. FIG. 10 is a diagram for describing processing performed by the likelihood image synthesizer 13e according to the present embodiment. FIG. 11 is a diagram illustrating an example of an image synthesis method according to an identification target stored in the image synthesis method data table D2.

As illustrated in FIG. 9, the target identifier 13c according to the present embodiment performs segmentation processing based on each structure type on the ultrasonic images sequentially generated by the signal processor 12 to generate a likelihood image indicating an existence region of a target. That is, for example, the target identifier 13c performs identification processing on the ultrasonic image B generated by the ultrasonic scanning using the ultrasonic beam with the steer angle of 0 degrees to generate a likelihood image B1, performs identification processing on the ultrasonic image A generated by the ultrasonic scanning using the ultrasonic beam with the steer angle of −θ degrees to generate a likelihood image A1, and performs identification processing on the ultrasonic image C generated by the ultrasonic scanning using the ultrasonic beam with the steer angle of +θ degrees to generate a likelihood image C1. Here, the identification models D1 applied to the ultrasonic image A, the ultrasonic image B, and the ultrasonic image C may be the same or may be different optimized for each steer angle.

According to the processing of the target identifier 13c, since there is no influence of a motion artifact caused by space compound synthesis, the target identifier 13c can generate the likelihood image A1, the likelihood image B1, and the likelihood image C1 in which likelihoods of target regions are accurately calculated from the ultrasonic image A, the ultrasonic image B, and the ultrasonic image C, respectively.

Furthermore, according to the processing of the target identifier 13c, a structure having acoustic reflection anisotropy with respect to an ultrasonic beam, such as a puncture needle QT, can also be identified with high accuracy in any of the likelihood image A1, the likelihood image B1, or the likelihood image C1. In FIG. 9, a direction of the ultrasonic beam is orthogonal to an extending direction of the puncture needle QT at the time of the ultrasonic beam having the steer angle of +θ degrees, and the puncture needle QT is clearly visualized in the ultrasonic image C and the likelihood image C1.

Furthermore, according to the processing of the target identifier 13c, a structure existing at an end of an image can also be identified with high accuracy in any of the likelihood image A1, the likelihood image B1, or the likelihood image C1. For example, in FIG. 9, nerve tissue HT is clearly visualized in the ultrasonic image A, and the likelihood image A1 in which the likelihood of the target region is accurately calculated can be generated.

Then, the likelihood image synthesizer 13e according to the present embodiment synthesizes the plurality of likelihood images A1, B1, and C1 using the image synthesis method set for each structure type stored in the image synthesis method data table D2, as illustrated in FIG. 11, to generate a space compound likelihood image Sy1.

For example, when the target to be identified is a structure having acoustic reflection anisotropy with respect to an ultrasonic beam (for example, the puncture needle QT), the likelihood image synthesizer 13e selects a likelihood having a maximum value from among the likelihoods of the plurality of likelihood images A1, B1, and C1 to be synthesized or selectively adds likelihoods of a threshold value or more among the likelihoods of the plurality of likelihood images A1, B1, and C1 to be synthesized for each pixel region, thereby image synthesizing the plurality of likelihood images A1, B1, and C1. In addition, when the target to be identified is a structure having no acoustic reflection anisotropy with respect to the ultrasonic beam (for example, the nerve tissue HT), the likelihood image synthesizer 3e averages the likelihoods of the plurality of likelihood images A1, B1, and C1 to be synthesized for each pixel region, thereby image synthesizing the plurality of likelihood images A1, B1, and C1.

Note that examples of the structure having acoustic reflection anisotropy with respect to the ultrasonic beam include a puncture needle and a fascia, and examples of the structure having no acoustic reflection anisotropy with respect to the ultrasonic beam include nerve tissue and muscle tissue.

In a case where there is one type of target, the likelihood image synthesizer 13e may synthesize the likelihood images A1, B1, and C1 with respect to an entire region of the likelihood image by using one type of image synthesis method corresponding to a structure type of the target. On the other hand, in a case where there is a plurality of types of targets (that is, in a case where a likelihood image indicating likelihood distributions of the plurality of types of targets is generated), the likelihood image synthesizer 13e may synthesize a plurality of likelihood images for each pixel region of the likelihood image by using an image synthesis method according to a type of the target present in the pixel region.

Note that FIG. 9 illustrates an aspect in which two types of targets of the nerve tissue HT and the puncture needle QT are set as targets to be identified. Then, each pixel value in a region of the nerve tissue HT of the space compound likelihood image Sy1 is calculated as an average value of the likelihoods of the likelihood images A1, B1, and C1, and each pixel value in a region of the puncture needle QT of the space compound likelihood image Sy1 is calculated by selecting a maximum value from the likelihoods of the likelihood images A1, B1, and C1.

According to the processing of the likelihood image synthesizer 13e, since the space compound likelihood image Sy1 can be generated by synthesizing the likelihood image A1, the likelihood image B1, and the likelihood image C1 in which the likelihoods of the target regions are calculated with high accuracy, the likelihood image synthesizer 13e can construct a highly accurate likelihood image (that is, a likelihood distribution) in which influence of a motion artifact is suppressed. This is because it is possible to avoid difficulty of identifying the target from the blurred space compound ultrasonic image due to the influence of the motion artifact by using the identification model.

Furthermore, according to the processing of the likelihood image synthesizer 13e, the plurality of likelihood images A1, B1, and C1 are synthesized using the image synthesis method set for each structure type to generate the space compound likelihood image Sy1. Therefore, for example, information of a likelihood image in which a structure having acoustic reflection anisotropy with respect to the ultrasonic beam (here, the puncture needle QT) is clearly visualized among the likelihood image A1, the likelihood image B1, or the likelihood image C1 clearly appears on the space compound likelihood image Sy1. That is, it is possible to clarify the existence region of the target on the space compound likelihood image Sy1.

In addition, according to the processing of the likelihood image synthesizer 13e, since the likelihood images A1, B1, and C1 are generated from the ultrasonic images A, B, and C, respectively, and then synthesized, the target present at the end of the image can also be identified with high accuracy. This is because the entire structure existing at the end of the image is reflected in at least one of the ultrasonic image A, the ultrasonic image B, or the ultrasonic image C, and the target is identified with high accuracy in at least one of the likelihood image A1, the likelihood image B1, and the likelihood image C1 generated from the ultrasonic image A, the ultrasonic image B, and the ultrasonic image C.

Note that, before synthesizing the likelihood image A1, the likelihood image B1, and the likelihood image C1, the likelihood image synthesizer 13e preferably removes noise included in each of the likelihood image A1, the likelihood image B1, and the likelihood image C1 on the basis of a change in information regarding the likelihood (for example, a target likelihood and a likelihood distribution) obtained from temporally consecutive ultrasonic images. At this time, for example, the likelihood image synthesizer 13e preferably preforms noise processing while being divided into a temporal change of the likelihood image B1 obtained from the ultrasonic image B generated by the ultrasonic scanning using the ultrasonic beam with the steer angle of 0 degrees, a temporal change of the likelihood image A1 obtained from the ultrasonic image A generated by the ultrasonic scanning using the ultrasonic beam with the steer angle of −θ degrees, and a temporal change of the likelihood image C1 obtained from the ultrasonic image C generated by the ultrasonic scanning using the ultrasonic beam with the steer angle of +θ degrees.

In this case, the likelihood image synthesizer 13e can remove noise included in the likelihood image by applying, for example, moving average filter processing or median filter processing in a time axis direction. In this case, a region where a change (steepness) in the information regarding the likelihood exceeds a preset threshold may be detected as a noise region, and the noise removal processing may be performed only for this noise region.

Furthermore, when synthesizing the likelihood image A1, the likelihood image B1, and the likelihood image C1, the likelihood image synthesizer 13e may perform normalization processing on each of the likelihood image A1, the likelihood image B1, and the likelihood image C1 as preprocessing.

The display image generation unit 13f applies the space compound likelihood image Sy1 generated by the likelihood image synthesizer 13e as an enhancement map of the target region in the space compound ultrasonic image Sy generated by the ultrasonic image synthesizing unit 13b.

For example, the display image generation unit 13f superimposes the space compound likelihood image Sy1 generated by the likelihood image synthesizer 13e on the space compound ultrasonic image Sy generated by the ultrasonic image synthesizing unit 13b, and outputs the superimposed image to the display unit 14.

At this time, the display image generation unit 13f may synthesize the space compound ultrasonic image Sy and the space compound likelihood image Sy1 using, for example, the color map illustrated in FIG. 1. For example, the display image generation unit 13f adds color information (hue, saturation, and lightness) to each pixel of the space compound ultrasonic image Sy on the basis of a pixel value of the space compound ultrasonic image Sy and a pixel value of the space compound likelihood image Sy1 that are in a positional relationship corresponding to each other in the image, and changes at least one of the hue, saturation, and lightness of the space compound ultrasonic image Sy to generate a display image to be provided for the user.

Note that the display image generation unit 13f may display the space compound ultrasonic image Sy and the space compound likelihood image Sy1 side by side instead of superimposing the space compound likelihood image Sy1 generated by the likelihood image synthesizer 13e on the space compound ultrasonic image Sy generated by the ultrasonic image synthesizing unit 13b

[Effects]

As described above, an ultrasonic diagnostic apparatus 1 according to the present embodiment includes:
a transmitter/receiver 11 that causes an ultrasonic probe 20 to transmit and receive an ultrasonic beam;
a signal processor 12 that generates an ultrasonic image on the basis of a reception signal acquired from the ultrasonic probe 20;
a target identifier 13c that performs segmentation processing based on a structure type on the ultrasonic image to generate a likelihood image indicating an existence region of a target in the ultrasonic image; and
a likelihood image synthesizer 13e that synthesizes the likelihood images of the plurality of ultrasonic images generated by ultrasonic scanning using ultrasonic beams having steer angles different from each other to generate a space compound likelihood image.

Therefore, according to the ultrasonic diagnostic apparatus 1 according to the present embodiment, it is possible to construct a highly accurate likelihood image (that is, a likelihood distribution) in which influence of a notion artifact is suppressed. In addition, this makes it possible to identify a target appearing at an end of an image with high accuracy. That is, it is possible to further clarify the existence region of the target on the likelihood image (here, a space compound likelihood image Sy1).

Further, in the ultrasonic diagnostic apparatus 1 according to the present embodiment, in particular, the likelihood image synthesizer 13e synthesizes the plurality of likelihood images using an image synthesis method set for each structure type to generate the space compound likelihood image.

Therefore, according to the ultrasonic diagnostic apparatus 1 according to the present embodiment, a structure having acoustic reflection anisotropy with respect to the ultrasonic beam (a puncture needle, a fascia, or the like) can also be identified with high accuracy. As a result, it is possible to further clarify the existence region of the target on the likelihood image (here, the space compound likelihood image Sy1).

### Modified Examples

In the above embodiment, the likelihood image synthesizer 13e synthesizes the plurality of likelihood images A1, B1, and C1 using the image synthesis method set for each structure type stored in advance in the image synthesis method data table D2 to generate the space compound likelihood image Sy1 (see FIG. 11).

However, the image synthesis method in the likelihood image synthesizer 13e may be settable by a user. This enables more flexible processing.

For example, the likelihood image synthesizer 13e may allow the user to set the image synthesis method for each target appearing in the ultrasonic image or for each pixel region of the ultrasonic image. As a result, for example, it is also possible to set such that a maximum value of likelihoods of the likelihood images A1, B1, and C1 is selected for the target appearing at an end of the image, and on the other hand, an average value of the likelihoods of the likelihood images A1, B1, and C1 is calculated for the target appearing at the center of the image. Note that, in this case, a user interface image may be displayed on the display unit 14 so that the user can selectively set the image synthesis method for a predetermined pixel region from the ultrasonic image.

Although specific examples of the present invention have been described in detail above, these are merely examples, and do not limit the scope of claims. The technology described in the claims includes various modifications and changes of the specific examples exemplified above.

According to the ultrasonic diagnostic apparatus of the present disclosure, it is possible to improve accuracy of a likelihood image indicating a target region in ultrasonic image diagnosis using a space compound method.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus comprising:
a transmitter/receiver that causes an ultrasonic probe to transmit and receive an ultrasonic beam;
a signal processor that generates an ultrasonic image on a basis of a reception signal acquired from the ultrasonic probe;
a target identifier that performs segmentation processing based on a structure type on the ultrasonic image to generate a likelihood image indicating an existence region of a target in the ultrasonic image; and
a likelihood image synthesizer that synthesizes the likelihood images of a plurality of the ultrasonic images generated by ultrasonic scanning using the ultrasonic beams having steer angles different from each other to generate a space compound likelihood image.

2. The ultrasonic diagnostic apparatus according to claim 1, wherein
the space compound likelihood image is applied as an enhancement map of a target region in a space compound ultrasonic image generated by synthesizing the plurality of ultrasonic images generated by the ultrasonic scanning using the ultrasonic beams having the steer angles different from each other.

3. The ultrasonic diagnostic apparatus according to claim 1, wherein
the likelihood image synthesizer synthesizes a plurality of the likelihood images by using an image synthesis method set for the structure type to generate the space compound likelihood image.

4. The ultrasonic diagnostic apparatus according to claim 3, wherein
when a plurality of types of the targets is set,
the likelihood image synthesizer synthesizes the plurality of likelihood images for each pixel region of the likelihood image by using an image synthesis method according to a type of the target existing in the pixel region to generate the space compound likelihood image.

5. The ultrasonic diagnostic apparatus according to claim 3, wherein
the likelihood image synthesizer
synthesize the plurality of likelihood images by selecting a likelihood having a maximum value among the likelihoods of the plurality of likelihood images to be synthesized, or selectively adding likelihoods of a threshold value or more among the likelihoods of the plurality of likelihood images to be synthesized for each pixel region, when the target to be identified is a first structure having acoustic reflection anisotropy with respect to the ultrasonic beam, and
synthesizes the plurality of likelihood images by averaging the likelihoods of the plurality of likelihood images to be synthesized for each pixel region, when the target to be identified is a second structure having no acoustic reflection anisotropy with respect to the ultrasonic beam.

6. The ultrasonic diagnostic apparatus according to claim 5, wherein
the first structure includes a puncture needle, and
the second structure includes nerve tissue.

7. The ultrasonic diagnostic apparatus according to claim 1, wherein
the likelihood image synthesizer synthesizes a plurality of the likelihood images by using an image synthesis method set by a user to generate the space compound likelihood image.

8. The ultrasonic diagnostic apparatus according to claim 1, wherein
the target identifier performs segmentation processing based on the structure type on each of the plurality of ultrasonic images by using an identification model learned by machine learning.

9. The ultrasonic diagnostic apparatus according to claim 8, wherein
the identification model is a neural network.

10. A method for controlling an ultrasonic diagnostic apparatus comprising:
causing an ultrasonic probe to transmit and receive an ultrasonic beam;
generating an ultrasonic image on a basis of a reception signal acquired from the ultrasonic probe;
performing segmentation processing based on a structure type on the ultrasonic image to generate a likelihood image indicating an existence region of a target in the ultrasonic image; and
synthesizing the likelihood images of a plurality of the ultrasonic images generated by ultrasonic scanning using the ultrasonic beams laving steer angles different from each other to generate a space compound likelihood image.

11. A non-transitory recording medium storing a computer readable control program for an ultrasonic diagnostic apparatus causing a computer to perform:
causing an ultrasonic probe to transmit and receive an ultrasonic beam;
generating an ultrasonic image on a basis of a reception signal acquired from the ultrasonic probe;
performing segmentation processing based on a structure type on the ultrasonic image to generate a likelihood image indicating an existence region of a target in the ultrasonic image; and
synthesizing the likelihood images of a plurality of the ultrasonic images generated by ultrasonic scanning using the ultrasonic beams having steer angles different from each other to generate a space compound likelihood image.
